(19) United States
(12) Patent Application Publication
(10) Pub. No.: US 2024/0294900 A1
(43) Pub. Date: Sep. 5, 2024

(54) Common Cold Coronavirus T Cell Epitopes, Methods and Uses Thereof

(71) Applicants: La Jolla Institute for Immunology, La Jolla, CA (US); J. Craig Venter Institute, La Jolla, CA (US)

(72) Inventors: Alessandro Sette, La Jolla, CA (US); Alba Grifoni, La Jolla, CA (US); Alison Tarke, La Jolla, CA (US); Yun Zhang, San Diego, CA (US); Richard Scheuerman, La Jolla, CA (US)

(21) Appl. No.: 18/403,239

(22) Filed: Jan. 3, 2024

Related U.S. Application Data
(63) Continuation-in-part of application No. PCT/US2022/042741, filed on Sep. 7, 2022.
(60) Provisional application No. 63/241,824, filed on Sep. 8, 2021.

(51) Int. Cl. C12N 15/10

(52) U.S. Cl. CPC C12N 15/1037; C12N 15/1055

(57) ABSTRACT

The present invention includes compositions and methods for detecting the presence of: a coronavirus or an immune response relevant to a coronavirus infection including T cells responsive to one or more coronavirus peptides or proteins comprising, consisting of, or consisting essentially of: one or more amino acid sequences selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); a pool of 2 or more peptides selected from the amino acid sequences set forth in Tables 1 or 2 (SEQ ID NOS: 1 to 166); or a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof. The invention further provides vaccines, diagnostics, therapies, and kits, comprising such proteins or peptides.

### CROSS-REFERENCE TO RELATED APPLICATIONS

This application claims priority to U.S. Provisional Application Ser. No. 63/241,824, filed Sep. 8, 2021, and is a Continuation-in-Part of and claims priority to Patent Cooperation Treaty filing No. PCT/US22/42741, filed Sep. 7, 2022, the entire contents of each of which are incorporated herein by reference.

### STATEMENT OF FEDERALLY FUNDED RESEARCH

This invention was made with government support under Contact No. HHS75N93019C00076 and P01 AI168347 awarded by the National Institutes of Health. The government has certain rights in the invention.

### TECHNICAL FIELD OF THE INVENTION

The present invention relates in general to the field of peptides that are T cell epitopes for coronavirus, and more particularly, to compositions and methods for the prevention, treatment, diagnosis of coronavirus, including SARS-COV-2 and symptoms thereof, including vaccines, kits and other uses of and/or compositions comprising such T cell epitopes for use in detecting, treating, diagnosing, and/or characterizing common cold coronavirus T cell epitope specific responses in infection as well as following vaccination.

### INCORPORATION-BY-REFERENCE OF MATERIALS FILED ON COMPACT DISC

The present application includes a Sequence Listing which has been submitted in XML format via EFS-Web and is hereby incorporated by reference in its entirety. Said XML copy, created on Mar. 21, 2024 is named LJII1026CIP.xml and is 208,896 bytes in size.

### BACKGROUND OF THE INVENTION

Without limiting the scope of the invention, its background is described in connection with coronaviruses.

As of September 2021, SARS-COV-2 infections are associated with more than 4.5 million deaths and over 215 million cases worldwide, and over 40 million cases in the United States alone (https://coronavirus.jhu.edu/map.html). The severity of the associated Coronavirus Disease 2019 (COVID-19) ranges from asymptomatic or mild self-limiting disease, to severe pneumonia and acute respiratory distress syndrome (WHO; https://www.who.int/publications/i/item/clinical-management-of-covid-19). The present inventors and others have started to delineate the role of SARS-COV-2-specific T cell immunity in COVID-19 clinical outcomes (Altmann and Boyton, 2020; Braun et al., 2020; Grifoni et al., 2020; Le Bert et al., 2020; Meckiff et al., 2020; Rydyznski Moderbacher et al., 2020; Sekine et al., 2020; Weiskopf et al., 2020). A growing body of evidence points to a key role for SARS-COV-2-specific T cell responses in COVID-19 disease resolution and modulation of disease severity (Rydyznski Moderbacher et al., 2020; Schub et al., 2020; Weiskopf et al., 2020). Milder cases of acute COVID-19 were associated with coordinated antibody, CD4+ and CD8+ T cell responses, whereas severe cases correlated with a lack of coordination of cellular and antibody responses, and delayed kinetics of adaptive responses (Rydyznski Moderbacher et al., 2020; Weiskopf et al., 2020). Now, the emergence of SARS-CoV-2 variants highlights the better need to better understand adaptive immune responses to this virus.

The emergence of several new SARS-COV-2 variants remains of immediate concern to the medical and scientific community. Several other Corona Viruses (CoV) are known to infect bats and other animal species and are of concern as they could jump to human hosts and foster a new pandemic. For all the above reasons it is important to explore vaccine concepts that might be able to provide large spectrum immunity against several CoV viral species. The inventors disclose the discovery of T cell epitopes that provide broad protective immunity to multiple CoV species.

### SUMMARY OF THE INVENTION

As embodied and broadly described herein, an aspect of the present disclosure relates to a composition comprising: one or more peptides or proteins, comprising, consisting of, or consisting essentially of an amino acid sequence selected from the sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); a pool of 2 or more or more peptides comprising, consisting of, or consisting essentially of amino acid sequences selected from those sequences set forth in Tables 1 or 2 (SEQ ID NOS: 1 to 166); or a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof. In one aspect, the one or more peptides or proteins comprises, or wherein the fusion protein comprises 2 or more or more amino acid sequences selected from those sequences set forth in Tables 1 or 2 (SEQ ID NOS: 1 to 166), or a subsequence, portion, homologue, variant or derivative thereof. In another aspect, the composition comprises one or more coronavirus peptide amino acid sequences selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from those sequences set forth in Tables 1 or 2 (SEQ ID NOS: 1 to 166). In another aspect, the peptide or protein comprises a coronavirus T cell epitope. In another aspect, the one or more peptides or proteins comprises a coronavirus CD8+ or CD4+ T cell epitope. In another aspect, the coronavirus is NL63 or OC43 and the T cell epitope is not conserved in another coronavirus. In another aspect, the coronavirus is NL63 or OC43 and the T cell epitope is conserved in another coronavirus, including SARS-COV-2. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a coronavirus. In another aspect, the one or more peptides or proteins that elicits, stimulates, induces, promotes, increases or enhances the T cell response to the coronavirus is a coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: peptides or proteins comprising, consisting of, or consisting essentially of: one or more amino acid sequences selected from those sequences set forth in Tables 1 or 2 (SEQ ID NOS: 1 to 166), concatemers, subsequences, portions, homologues, variants or derivatives thereof; a fusion protein comprising one or more amino acid sequences selected from those sequences set forth in Tables 1 or 2 (SEQ ID NOS: 1 to 166); or a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof.

As embodied and broadly described herein, an aspect of the present disclosure relates to a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 or 2 (SEQ ID NOS: 1 to 166), in a groove of the MHC monomer or multimer.

As embodied and broadly described herein, an aspect of the present disclosure relates to a composition comprising: one or more peptides or proteins comprising, consisting of, or consisting essentially of an amino acid sequence selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); a pool of 2 or more peptides selected from those sequences set forth in Tables 1 or 2 (SEQ ID NOS: 1 to 166); a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof. In one aspect, the one or more peptides or proteins comprises, or wherein the fusion protein comprises, 2 or more amino acid sequences selected from those sequences set forth in Tables 1 or 2 (SEQ ID NOS: 1 to 166), or a subsequence, portion, homologue, variant or derivative thereof. In another aspect, the protein or peptide comprises a coronavirus T cell epitope. In certain aspects, the coronavirus is NL63, OC43, and/or SARS-COV-2. In another aspect, the one or more peptides or proteins comprises a coronavirus CD8+ or CD4+ T cell epitope. In another aspect, the T cell epitope is not conserved in another coronavirus. In another aspect, the T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to SARS-COV-2. In another aspect, the one or more peptides or proteins that elicits, stimulates, induces, promotes, increases or enhances the T cell response to SARS-COV-2 is a NL63 or OC43 spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

As embodied and broadly described herein, an aspect of the present disclosure relates to a composition comprising monomers or multimers of: one or more peptides or proteins comprising, consisting of, or consisting essentially of: one or more SARS-COV-2 amino acid sequences selected from those sequences set forth in Tables 1 or 2 (SEQ ID NOS: 1 to 166), concatemers, subsequences, portions, homologues, variants or derivatives thereof; a fusion protein comprising one or more amino acid sequences selected from those sequences set forth in Tables 1 or 2 (SEQ ID NOS: 1 to 166); or a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof.

As embodied and broadly described herein, an aspect of the present disclosure relates to a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 or 2 (SEQ ID NOS: 1 to 166), in a groove of the (MHC) monomer or multimer.

As embodied and broadly described herein, an aspect of the present disclosure relates to a method for detecting the presence of: (i) a coronavirus or (ii) an immune response relevant to coronavirus infections, vaccines or therapies, including T cells responsive to one or more coronavirus peptides, comprising: providing one or more proteins or peptides for detection of an amount or a relative amount of, and/or the activity of, and/or the state of antigen-specific T-cells; contacting a biological sample suspected of having coronavirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in Tables 1 or 2 (SEQ ID NOS: 1 to 166), or comprise a pool of 2 or more or more amino acid sequences set forth in Tables 1 or 2 (SEQ ID NOS: 1 to 166). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from Tables 1 or 2. In another aspect, the detecting the amount or a relative amount of, and/or activity of antigen-specific T-cells comprises indirect detection and/or direct detection. In another aspect, the method of detecting an immune response relevant to the coronavirus comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the coronavirus. In another aspect, the protein or peptide comprises a CD8+ or CD4+ T cell epitope. In another aspect, the T cell epitope is not conserved in another coronavirus. In another aspect, the T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Tables 1 or 2 (SEQ ID NOS: 1 to 166), or a subsequence, portion, homologue, variant or derivative thereof. In another aspect, the method further comprises detecting the presence or amount of the one or more peptides in a biological sample, or a response thereto, which is diagnostic of a coronavirus infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

As embodied and broadly described herein, an aspect of the present disclosure relates to a method for detecting the presence of: (i) SARS-COV-2 or (ii) an immune response relevant to SARS-COV-2 infections, vaccines or therapies, including T cells responsive to one or more SARS-COV-2 peptides, comprising: providing one or more proteins or peptides for detection of an amount or a relative amount of, and/or the activity of, and/or the state of antigen-specific T-cells; contacting a biological sample suspected of having SARS-COV-2-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in Tables 1 or 2 (SEQ ID NOS: 1 to 166), or comprise a pool of 2 or more amino acid sequences set forth in those sequences set forth in Tables 1 or 2 (SEQ ID NOS: 1 to 166). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from those sequences set forth in Tables 1 or 2 (SEQ ID NOS: 1 to 166). In another aspect, detecting the amount or a relative amount of, and/or activity of antigen-specific T-cells comprises indirect detection and/or direct detection. In another aspect, detecting an immune response relevant to SARS-COV-2 comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of SARS-COV-2. In another aspect, the protein or peptide comprises a SARS-COV-2 CD8+ or CD4+ T cell epitope. In another aspect, the SARS-COV-2 T cell epitope is not conserved in another coronavirus. In another aspect, the SARS-COV-2 T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Tables 1 or 2 (SEQ ID NOS: 1 to 166), or a subsequence, portion, homologue, variant or derivative thereof. In another aspect, the method further comprises detecting the presence or amount of the one or more peptides in a biological sample, or a response thereto, which is diagnostic of a SARS-COV-2 infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

As embodied and broadly described herein, an aspect of the present disclosure relates to a method detecting a coronavirus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with coronavirus. In one aspect, the sample comprises T cells. In another aspect, the response comprises inducing, increasing, promoting or stimulating anti-coronavirus activity of T cells. In another aspect, the T cells are CD8+ or CD4+ T cells. In another aspect, the method comprises determining whether the subject has been infected by or exposed to the coronavirus more than once by determining if the subject elicits a secondary T cell immune response profile that is different from a primary T cell immune response profile. In another aspect, the method further comprises diagnosing a coronavirus infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a coronavirus. In another aspect, the method is conducted three or more days following the date of suspected infection by or exposure to a coronavirus.

As embodied and broadly described herein, an aspect of the present disclosure relates to a method detecting SARS-COV-2 infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with SARS-CoV-2. In another aspect, the sample comprises T cells. In another aspect, the response comprises inducing, increasing, promoting or stimulating anti-SARS-COV-2 activity of T cells. In another aspect, the T cells are CD8+ or CD4+ T cells. In another aspect, the method comprises determining whether the subject has been infected by or exposed to SARS-COV-2 more than once by determining if the subject elicits a secondary T cell immune response profile that is different from a primary T cell immune response profile. In another aspect, the method further comprises diagnosing a SARS-COV-2 infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to SARS-COV-2. In another aspect, the method is conducted three or more days following the date of suspected infection by or exposure to a coronavirus.

As embodied and broadly described herein, an aspect of the present disclosure relates to a kit for the detection of coronavirus or an immune response to coronavirus in a subject comprising, consisting of or consisting essentially of: one or more T cells that specifically detect the presence of: one or more amino acid sequences selected from those sequences set forth in Tables 1 or 2 (SEQ ID NOS: 1 to 166), or a subsequence, portion, homologue, variant or derivative thereof; or a fusion protein comprising one or more amino acid sequences selected from those sequences set forth in Tables 1 or 2 (SEQ ID NOS: 1 to 166); or a pool of 2 or more or more peptides selected from the amino acid sequences set forth in Tables 1 or 2 (SEQ ID NOS: 1 to 166). In one aspect, the one or more amino acid sequences are selected from a coronavirus T cell epitope set forth in Tables 1 or 2 (SEQ ID NOS: 1 to 166). In another aspect, the composition comprises: one or more amino acid sequences selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from the amino acid sequences set forth in those sequences set forth in Tables 1 or 2 (SEQ ID NOS: 1 to 166). In another aspect, the amino acid sequence comprises a coronavirus CD8+ or CD4+ T cell epitope. In another aspect, the T cell epitope is not conserved in another coronavirus. In another aspect, the T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to coronavirus.

As embodied and broadly described herein, an aspect of the present disclosure relates to a kit for the detection of SARS-COV-2 or an immune response to SARS-COV-2 in a subject comprising, consisting of or consisting essentially of: one or more T cells that specifically detect the presence of: one or more amino acid sequences selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from the amino acid sequences set forth in those sequences set forth in Tables 1 or 2 (SEQ ID NOS: 1 to 166). In another aspect, the amino acid sequence comprises a SARS-CoV-2 CD8+ or CD4+ T cell epitope. In another aspect, the SARS-COV-2 T cell epitope is not conserved in another coronavirus. In another aspect, the SARS-COV-2 T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) SARS-COV-2 or (ii) an immune response relevant to SARS-COV-2 infections, vaccines or therapies, including T cells responsive to SARS-COV-2. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to SARS-COV-2.

As embodied and broadly described herein, an aspect of the present disclosure relates to a method of stimulating, inducing, promoting, increasing, or enhancing an immune response against a coronavirus in a subject, comprising: administering a composition of one or more proteins, peptides, multimers or a polynucleotide that expresses the protein, peptide or multimers, in an amount sufficient to stimulate, induce, promote, increase, or enhance an immune response against the coronavirus in the subject. In another aspect, the immune response provides the subject with protection against a coronavirus infection or pathology, or one or more physiological conditions, disorders, illnesses, diseases or symptoms caused by or associated with coronavirus infection or pathology. In another aspect, the immune response is specific to: one or more SARS-COV-2 peptides selected from the amino acid sequences set forth in Tables 1 or 2 (SEQ ID NOS: 1 to 166), or a subsequence, portion, homologue, variant or derivative thereof.

As embodied and broadly described herein, an aspect of the present disclosure relates to a method of stimulating, inducing, promoting, increasing, or enhancing an immune response against SARS-COV-2 in a subject, comprising: administering a composition of proteins, peptides, multimers or a polynucleotide that expresses the protein, peptide or multimers, in an amount sufficient to stimulate, induce, promote, increase, or enhance an immune response against SARS-COV-2 in the subject. In one aspect, the immune response provides the subject with protection against a SARS-COV-2 infection or pathology, or one or more physiological conditions, disorders, illnesses, diseases or symptoms caused by or associated with SARS-CoV-2 infection or pathology. In another aspect, the immune response is specific to: one or more coronavirus peptides selected from the amino acid sequences set forth in those sequences set forth in Tables 1 or 2 (SEQ ID NOS: 1 to 166), or a subsequence, portion, homologue, variant or derivative thereof.

As embodied and broadly described herein, an aspect of the present disclosure relates to a method of stimulating, inducing, promoting, increasing, or enhancing an immune response against SARS-COV-2 in a subject, comprising: administering to a subject an amount of a protein or peptide comprising, consisting of or consisting essentially of an amino acid sequence of the coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Tables 1 or 2 (SEQ ID NOS: 1 to 166) or a subsequence, portion, homologue, variant or derivative thereof, in an amount sufficient to prevent, stimulate, induce, promote, increase, immunize against, or enhance an immune response against SARS-COV-2 in the subject. In one aspect, the immune response provides the subject with protection against SARS-COV-2 infection or pathology, or one or more physiological conditions, disorders, illnesses, diseases or symptoms caused by or associated with SARS-COV-2 infection or pathology.

As embodied and broadly described herein, an aspect of the present disclosure relates to a method of treating, preventing, or immunizing a subject against SARS-COV-2 infection, comprising administering to a subject an amount of a protein or peptide comprising, consisting of, or consisting essentially of an amino acid sequence of a coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two amino acid sequences selected from Tables 1 or 2 (SEQ ID NOS: 1 to 166) or a subsequence, portion, homologue, variant or derivative thereof, in an amount sufficient to treat, prevent, or immunize the subject for SARS-COV-2 infection, wherein the protein or peptide comprises or consists of a coronavirus T cell epitope that elicits, stimulates, induces, promotes, increases, or enhances an anti-SARS-COV-2 T cell immune response. In one aspect, the one or more amino acid sequences are selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from the amino acid sequences set forth in those sequences set forth in Tables 1 or 2 (SEQ ID NOS: 1 to 166). In one aspect, the anti-SARS-CoV-2 T cell response is a CD8+, a CD4+ T cell response, or both. In another aspect, the T cell epitope is conserved across two or more clinical isolates of SARS-COV-2, two or more circulating forms of SARS-COV-2, or two or more coronaviruses. In another aspect, the SARS-CoV-2 infection is an acute infection. In another aspect, the subject is a mammal or a human. In another aspect, the method reduces SARS-COV-2 viral titer, increases or stimulates SARS-CoV-2 viral clearance, reduces or inhibits SARS-COV-2 viral proliferation, reduces or inhibits increases in SARS-COV-2 viral titer or SARS-COV-2 viral proliferation, reduces the amount of a SARS-COV-2 viral protein or the amount of a SARS-COV-2 viral nucleic acid, or reduces or inhibits synthesis of a SARS-COV-2 viral protein or a SARS-COV-2 viral nucleic acid. In another aspect, the method reduces one or more adverse physiological conditions, disorders, illness, diseases, symptoms or complications caused by or associated with SARS-COV-2 infection or pathology. In another aspect, the method improves one or more adverse physiological conditions, disorders, illness, diseases, symptoms or complications caused by or associated with SARS-COV-2 infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to SARS-COV-2 infection or pathology. In another aspect, the protein or peptide, or a subsequence, portion, homologue, variant or derivative thereof, is administered prior to, substantially contemporaneously with or following exposure to or infection of the subject with SARS-COV-2. In another aspect, a plurality of SARS-COV-2 T cell epitopes are administered prior to, substantially contemporaneously with or following exposure to or infection of the subject with

SARS-COV-2. In another aspect, the protein or peptide, or a subsequence, portion, homologue, variant or derivative thereof is administered within 2-72 hours, 2-48 hours, 4-24 hours, 4-18 hours, or 6-12 hours after a symptom of SARS-COV-2 infection or exposure develops. In another aspect, the protein or peptide, or a subsequence, portion, homologue, variant or derivative thereof is administered prior to exposure to or infection of the subject with SARS-CoV-2. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-β, or IL-10, or an agonist or antagonist thereof.

In another embodiment, the present invention includes a method of treating, preventing, or immunizing a subject against SARS-COV-2 infection, comprising administering to a subject the composition of one or more proteins, peptides or multimers in an amount sufficient to treat, prevent, or immunize the subject for SARS-COV-2 infection. In one aspect, the SARS-COV-2 infection is an acute infection. In another aspect, the method reduces SARS-COV-2 viral titer, increases or stimulates SARS-COV-2 viral clearance, reduces or inhibits SARS-COV-2 viral proliferation, reduces or inhibits increases in SARS-COV-2 viral titer or SARS-COV-2 viral proliferation, reduces the amount of a SARS-COV-2 viral protein or the amount of a SARS-CoV-2 viral nucleic acid, or reduces or inhibits synthesis of a SARS-COV-2 viral protein or a SARS-COV-2 viral nucleic acid. In another aspect, the method reduces one or more adverse physiological conditions, disorders, illness, diseases, symptoms or complications caused by or associated with SARS-COV-2 infection or pathology. In another aspect, the method improves one or more adverse physiological conditions, disorders, illness, diseases, symptoms or complications caused by or associated with SARS-COV-2 infection or pathology. In another aspect, the symptom is fever or chills, cough, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to SARS-COV-2 infection or pathology. In another aspect, the composition is administered prior to, substantially contemporaneously with or following exposure to or infection of the subject with SARS-COV-2. In another aspect, the composition is administered prior to, substantially contemporaneously with or following exposure to or infection of the subject with SARS-COV-2. In another aspect, the composition is administered within 2-72 hours, 2-48 hours, 4-24 hours, 4-18 hours, or 6-12 hours after a symptom of SARS-COV-2 infection or exposure develops. In another aspect, the composition is administered prior to exposure to or infection of the subject with SARS-COV-2.

As embodied and broadly described herein, an aspect of the present disclosure relates to a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-COV-2. In another aspect, the immunodominant peptides are selected from 1, 2 or more peptides selected from the amino acid sequences set forth in Tables 1 or 2 (SEQ ID NOS: 1 to 166). In another aspect, the immunodominant peptides are selected from 1, 2 or more peptides selected from the amino acid sequences set forth in those sequences set forth in Tables 1 or 2 (SEQ ID NOS: 1 to 166).

As embodied and broadly described herein, an aspect of the present disclosure relates to a method of selecting an immunoprevalent or immunodominant peptide or protein of a virus comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-COV-2. In another aspect, the immunodominant peptides are selected from 1, 2 or more peptides selected from the amino acid sequences set forth in Tables 1 or 2 (SEQ ID NOS: 1 to 166). In another aspect, the immunodominant peptides are selected from 1, 2 or more peptides selected from the amino acid sequences set forth in those sequences set forth in Tables 1 or 2 (SEQ ID NOS: 1 to 166).

As embodied and broadly described herein, an aspect of the present disclosure relates to a polynucleotide that expresses one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more or more peptides comprising, consisting of, or consisting essentially of amino acid sequences selected from those sequences set forth in Tables 1 or 2 (SEQ ID NOS: 1 to 166). In one aspect, the vector comprises the polynucleotide of claim that expresses one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more or more peptides comprising, consisting of, or consisting essentially of amino acid sequences selected from those sequences set forth in Tables 1 or 2 (SEQ ID NOS: 1 to 166), a viral vector, or a host cell the comprises the same.

As embodied and broadly described herein, an aspect of the present disclosure relates to a polynucleotide that expresses one or more peptides or proteins comprising, consisting of, or consisting essentially of an amino acid sequence selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from those sequences set forth in Tables 1 or 2 (SEQ ID NOS: 1 to 166). In one aspect, the vector comprises the polynucleotide of claim that expresses one or more peptides or proteins comprising, consisting of, or consisting essentially of an amino acid sequence selected from those sequences set forth in Tables 1 or 2 (SEQ ID NOS: 1 to 166), or a subsequence, portion, homologue, variant or derivative thereof; a fusion protein comprising one or more amino acid sequences selected from those sequences set forth in Tables 1 or 2 (SEQ ID NOS: 1 to 166); or a pool of 2 or more peptides selected from those sequences set forth in Tables 1 or 2 (SEQ ID NOS: 1 to 166), a viral vector, or a host cell that comprises the same.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

### Example 1

The inventors have synthetized overlapping peptides spanning the entire proteome of NL63 and OC43, chosen as prototypes for alpha and beta coronaviruses. PBMCs from a collection of approximately 100 unexposed donors were screened, and following a previously described strategy (Tarke et al., Cell Rep Med 2021) the inventors mapped the epitopes recognized. The inventors also previously reported a meta-analysis of known SARS-COV2 T cell epitopes (Grifoni et al., Cell Host & Microbe 2021). In parallel, the degree of conservation of SARS-COV2, NL63 and OC43_T cell epitopes across different coronavirus species was determined.

SARS-COV2, NL63 and OC43 epitopes were used to elicit T cell lines from donors known to respond to the various epitopes. The aligned homolog peptides are synthesized corresponding to the experimentally defined epitopes from different coronaviruses of interest. Each epitope specific T cell line is tested for cross-reactivity with the corresponding homologous peptides using the approach described in (Mateus et al., Science 2020) to demonstrate coronavirus cross-reactivity. This activity establishes which epitopes can be used to elicit broadly cross-reactive T cell responses.

### Definitions

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy, or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or virus.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer or higher valency multimer. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-O) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing coronavirus infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting. The MHC class I or class II peptide multimers as described herein can also be used as therapeutic agents. The peptide and/or the MHC class I or class II peptide multimers of the present invention are suitable for treating or preventing a coronavirus infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” or “immunization” are used interchangeably and refer to a composition that can provide active acquired immunity to and/or therapeutic effect (e.g., treatment) of a particular disease or a pathogen. A vaccine or immunization typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines or immunizations can be prophylactic (e.g., preventing or ameliorating the effects of a future infection by any natural or pathogen) or therapeutic (e.g., reducing symptoms or aberrant conditions associated with infection). The administration of a vaccine or immunization is referred to as vaccination or immunization, respectively.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus).

The present invention provides nucleic acid molecules, specifically polynucleotides, primary constructs and/or mRNA that encode one or more polynucleotides that express one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof. A variety of well-known vectors can be used to deliver to cells the one or more polynucleotides or the peptides or proteins expressed by the polynucleotides, including but not limited to adenoviral vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalovirus, adenovirus, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenovirus, adeno-associated virus, herpes virus, vaccinia virus, retroviruses, or other viral vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from those sequences set forth in Tables 1 or 2 (SEQ ID NOS: 1 to 166), or a subsequence, portion, homologue, variant or derivative thereof, gene expression construct. Viral vectors which do not require that the target cell be actively dividing, such as adenoviral and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311 or 222, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof into the viral vector, along with another gene which encodes the ligand for a receptor on a specific target cell, for example, such that the vector is now target specific. Viral vectors can be made target specific by attaching, for example, a sugar, a glycolipid, or a protein. Targeting can also be accomplished by using an antibody to target the viral vector. Those of skill in the art will know of, or can readily ascertain without undue experimentation, specific polynucleotide sequences which can be inserted into the viral genome or attached to a viral envelope to allow target specific delivery of the viral vector containing the gene.

Since recombinant viruses are defective, they require assistance in order to produce infectious vector particles. This assistance can be provided, for example, by using helper cell lines that contain plasmids encoding all of the structural genes of the virus under the control of regulatory sequences within the viral genome. These plasmids are missing a nucleotide sequence which enables the packaging mechanism to recognize a polynucleotide transcript for encapsidation. These cell lines produce empty virions, since no genome is packaged. If a viral vector is introduced into such cells in which the packaging signal is intact, but the structural genes are replaced by other genes of interest, the vector can be packaged and vector virion produced.

Viral or non-viral approaches may also be employed for the introduction of one or more therapeutic polynucleotides that express the one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a coronavirus or interact with the proteins, peptides, and/or gene products of a coronavirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalovirus, simian virus 40, actin or adenovirus constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2): 79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a coronavirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1 or 2, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof. Peptide and protein T cell epitopes of the invention can stimulate the T cell responses that, for example, neutralize infectious agents, kill virus infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins. In embodiments, the virus is a coronavirus. Non-limiting examples of coronaviruses (CoV) from which T cell epitopes can be identified include, e.g., SARS-COV (SARS-COV-1), MERS-COV, and SARS-COV-2, but also betacoronaviruses, e.g., HCoV-OC43, HCoVHKUI, HCoV-229E and alphacoronaviuses such as HCoV-NL63, and/or other coronaviruses endemic in humans. The viral genome of coronaviruses encodes at least the following structure proteins, the spike (S), envelope (E), membrane (M), and nucleocapsid (N) proteins. The S glycoprotein is responsible for binding the host receptor via the receptor-binding domain (RBD) in its S1 subunit, as well as the subsequent membrane fusion and viral entry driven by its S2 subunit. Gene sequencing of SARS-COV-2 showed that this novel coronavirus, a betacoronavirus, is related to the MERS-COV and the SARS-COV. SARS-COV, MERS-COV, and SARS-COV-2 belong to the betacoronavirus genus and are highly pathogenic zoonotic viruses. Thus, the present invention can be used not only to determine antigenic peptides from the three highly pathogenic betacoronaviruses, but also low-pathogenicity betacoronaviruses, such as, HCoV-OC43, HCoVHKUI, HCoV-NL63 and HCoV-229E, are also endemic in humans. In certain specific embodiments, the coronavirus is SARS-COV-2, including novel mutants of SARS-COV-2 that include mutants from five clades (19A, 19B, 20A, 20B, and 20C) according to Nextstrain, in GISAID nomenclature which divides them into seven clades (L, O, V, S, G, GH, and GR), and/or PANGOLIN nomenclature which divides them into six major lineages (A, B, B.1, B.1.1, B.1.177, B.1.1.7). Notable mutations of SARS-COV-2 include, e.g., D614G, P681H, N501Y, 69-70del, P681H, Y453F, 69-70deltaHV, N501Y, K417N, E484K, N501Y, and E484K.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is coronavirus infection. In certain alternative embodiments, the disease is SARS-COV-2 infection. In still other embodiments, the disease is COVID-19.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a coronavirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵ Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized sepharose (TM), agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™M QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™M (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like., that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in Tables 1 or 2 (SEQ ID NOS: 1 to 166)) or polynucleotides encoding the same, covering different HLA Class II and/or MHC Class I alleles, capable of generating a treatment acting broadly on a population level are disclosed herein. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

TABLE 1

| | | | | SEQ ID |
|---|---|---|---|---|
| CD4+ | Megapool | Start | Sequence | NO: |
| OC43 | nsp2 | 266 | DGLEAYADKTLQEMK | 1 |
| OC43 | nsp2 | 276 | LQEMKALFPTWSQEL | 2 |
| OC43 | nsp2 | 291 | LFDVIVAWHVVRDPR | 3 |
| OC43 | nsp2 | 566 | KFSLETFTVCADGFM | 4 |
| OC43 | nsp2 | 726 | LSCFKIGRRRICLSG | 5 |
| OC43 | nsp2 | 736 | ICLSGRKIYEVERGL | 6 |
| OC43 | nsp2 | 746 | VERGLLHSSQLPLDV | 7 |
| OC43 | nsp3 | 1006 | EVLEDTLDDGPSVET | 8 |
| OC43 | nsp3 | 1276 | NVCFVKGDIIKVSKL | 9 |
| OC43 | nsp3 | 1521 | RTVKYFECTGGIDIC | 10 |
| OC43 | nsp3 | 1821 | FIGDKVGHYVHVKCE | 11 |
| OC43 | nsp3 | 1831 | HVKCEQSYQLYDASN | 12 |
| OC43 | nsp3 | 1841 | YDASNVKKVTDVTGK | 13 |
| OC43 | nsp3 | 2151 | LLFGWIKISADNKVI | 14 |
| OC43 | nsp3 | 2166 | YTTEIASKLTCKLVA | 15 |
| OC43 | nsp3 | 2216 | NVIFSDFYLPKIGFL | 16 |
| OC43 | nsp3 | 2221 | DFYLPKIGFLPTFVG | 17 |
| OC43 | nsp3 | 2231 | PTFVGKIAQWIKNTF | 18 |
| OC43 | nsp3 | 2561 | NLITTANTGTSVTET | 19 |
| OC43 | nsp3 | 2566 | ANTGTSVTETMFDVY | 20 |
| OC43 | HE | 176 | APQANSGDYYYKVEA | 21 |
| OC43 | M | 21 | FLKEWNFSLGIILLF | 22 |
| OC43 | M | 36 | ITIILQFGYTSRSMF | 23 |
| OC43 | M | 46 | SRSMFVYVIKMIILW | 24 |
| OC43 | M | 56 | MIILWLMWPLTIILT | 25 |
| OC43 | M | 91 | IVAIIMWIVYFVNSI | 26 |
| OC43 | M | 101 | FVNSIRLFIRTGSFW | 27 |
| OC43 | M | 111 | TGSFWSENPETNNLM | 28 |
| OC43 | M | 136 | RPIIEDYHTLTVTII | 29 |
| OC43 | M | 146 | TVTIIRGHLYIQGIK | 30 |
| OC43 | M | 156 | IQGIKLGTGYSLADL | 31 |
| OC43 | M | 166 | SLADLPAYMTVAKVT | 32 |
| OC43 | M | 216 | QKGSGMDTALLRNNI | 33 |
| OC43 | N | 31 | DQFRNVQTRGRRAQP | 34 |
| OC43 | N | 66 | FSGITQFQKGKEFEF | 35 |
| OC43 | N | 121 | PRWYFYYLGTGPHAK | 36 |
| OC43 | N | 126 | YYLGTGPHAKDQYGT | 37 |
| OC43 | N | 141 | DIDGVYWVASNQADV | 38 |
| OC43 | N | 336 | NLSGNPDEPQKDVYE | 39 |
| OC43 | N | 346 | KDVYELRYNGAIRFD | 40 |
| OC43 | N | 351 | LRYNGAIRFDSTLSG | 41 |
| OC43 | N | 356 | AIRFDSTLSGFETIM | 42 |
| OC43 | N | 361 | STLSGFETIMKVLNE | 43 |
| OC43 | N | 366 | FETIMKVLNENLNAY | 44 |
| OC43 | N | 376 | NLNAYQQQDGMMNMS | 45 |
| OC43 | N | 434 | LKKMDEPYTEDTSEI | 46 |
| OC43 | S | 111 | NTKVIKDRVMYSEFP | 47 |
| OC43 | S | 401 | TIDKFAIPNGRKVDL | 48 |
| OC43 | S | 621 | NTDIILGVCVNYDLY | 49 |
| OC43 | S | 656 | NLLYDSNGNLYGFRD | 50 |
| OC43 | S | 716 | TRQLQPINYFDSYLG | 51 |
| OC43 | S | 721 | PINYFDSYLGCVVNA | 52 |
| OC43 | S | 886 | NFNVDDINFSPVLGC | 53 |
| OC43 | S | 911 | RSAIEDLLFDKVKLS | 54 |
| OC43 | S | 1086 | DRLINGRLTALNAYV | 55 |
| OC43 | S | 1091 | GRLTALNAYVSQQLS | 56 |
| OC43 | S | 1096 | LNAYVSQQLSDSTLV | 57 |
| OC43 | S | 1101 | SQQLSDSTLVKFSAA | 58 |
| OC43 | S | 1106 | DSTLVKFSAAQAMEK | 59 |
| OC43 | S | 1146 | QNAPYGLYFIHFSYV | 60 |
| OC43 | S | 1231 | LNTSIPNLPDFKEEL | 61 |
| OC43 | S | 1246 | DQWFKNQTSVAPDLS | 62 |
| NL63 | nsp3 | 956 | SAMNVIGQHIKLPQF | 63 |
| NL63 | nsp3 | 981 | VSKPVMISQWPISND | 64 |
| NL63 | nsp3 | 986 | MISQWPISNDSNGCV | 65 |
| NL63 | nsp3 | 1016 | DSVREEVDIIEQPFE | 66 |
| NL63 | nsp3 | 1051 | GVRVLDQSDNNCWIS | 67 |
| NL63 | nsp3 | 1061 | NCWISTTLVQLQLTK | 68 |
| NL63 | nsp3 | 1066 | TTLVQLQLTKLLDDS | 69 |
| NL63 | nsp3 | 1076 | LLDDSIEMQLFKVGK | 70 |
| NL63 | nsp3 | 1091 | VDSIVQKCYELSHLI | 71 |
| NL63 | nsp3 | 1251 | EIEAGEVKPFAVYKN | 72 |
| NL63 | nsp3 | 1286 | VNAANENLLHGGGVA | 73 |
| NL63 | nsp3 | 1326 | KVGAGVMLECEKFNV | 74 |
| NL63 | nsp3 | 1331 | VMLECEKFNVFNVVG | 75 |
| NL63 | nsp3 | 1421 | DLTPVIDDVDVVKPF | 76 |
| NL63 | nsp3 | 1436 | RVEGNFSFFDCGVNA | 77 |
| NL63 | nsp3 | 1451 | LDGDIYLLFTNSILM | 78 |
| NL63 | nsp3 | 1546 | PAIDVLKKLLSSLTL | 79 |
| NL63 | nsp3 | 1551 | LKKLLSSLTLTVKFV | 80 |
| NL63 | nsp3 | 1561 | TVKFVVESNVMDVND | 81 |
| NL63 | nsp3 | 2161 | FYVNANGGTCFCNKH | 82 |
| NL63 | nsp3 | 2171 | FCNKHNFFCVNCDSF | 83 |
| NL63 | nsp12 | 4096 | RFKNADLKDGYFVIK | 84 |
| NL63 | nsp12 | 4136 | HDFFTWKDGRVIYGN | 85 |
| NL63 | nsp12 | 4196 | SKGWYDPVENEDIHR | 86 |
| NL63 | nsp12 | 4216 | GKIVARAMLKCVALC | 87 |
| NL63 | nsp12 | 4236 | KGVVGVLTLDNQDLN | 88 |
| NL63 | nsp12 | 4476 | KHFFFAQNGDAAVKD | 89 |
| NL63 | nsp12 | 4866 | YLPYPDPSRILSAGV | 90 |
| NL63 | nsp12 | 4886 | VKTDAVVLLERYVSL | 91 |
| NL63 | nsp12 | 4891 | VVLLERYVSLAIDAY | 92 |
| NL63 | nsp12 | 4896 | RYVSLAIDAYPLSKH | 93 |
| NL63 | M | 6 | VPLLEVYVHLRNWNF | 94 |
| NL63 | M | 11 | VYVHLRNWNFSWNLI | 95 |
| NL63 | M | 16 | RNWNFSWNLILTLFI | 96 |
| NL63 | M | 21 | SWNLILTLFIVVLQY | 97 |
| NL63 | M | 26 | LTLFIVVLQYGHYKY | 98 |
| NL63 | M | 31 | VVLQYGHYKYSRLLY | 99 |
| NL63 | M | 36 | GHYKYSRLLYGLKMS | 100 |
| NL63 | M | 56 | WPLVLALSIFDCFVN | 101 |
| NL63 | M | 66 | DCFVNFNVDWVFFGF | 102 |
| NL63 | M | 101 | RLWRRVKTFWAFNPE | 103 |
| NL63 | M | 131 | LPVMAAPTGVTLTLL | 104 |
| NL63 | M | 146 | SGVLLVDGHKIATRV | 105 |
| NL63 | M | 151 | VDGHKIATRVQVGQL | 106 |
| NL63 | M | 161 | QVGQLPKYVIVATPS | 107 |
| NL63 | M | 166 | PKYVIVATPSTTIVC | 108 |
| NL63 | M | 176 | TTIVCDRVGRSVNET | 109 |
| NL63 | M | 181 | DRVGRSVNETSQTGW | 110 |
| NL63 | M | 186 | SVNETSQTGWAFYVR | 111 |
| NL63 | M | 201 | AKHGDFSGVASQEGV | 112 |
| NL63 | N | 16 | FPPPSFYMPLLVSSD | 113 |
| NL63 | N | 21 | FYMPLLVSSDKAPYR | 114 |
| NL63 | N | 101 | VAKEGAKTVNTSLGN | 115 |
| NL63 | N | 181 | SSSDLVAAVTLALKN | 116 |
| NL63 | N | 186 | VAAVTLALKNLGFDN | 117 |
| NL63 | N | 306 | YTYKMLVAKDNKNLP | 118 |
| NL63 | N | 316 | NKNLPKFIEQISAFT | 119 |
| NL63 | S | 226 | IPNGFPENNWELLTN | 120 |
| NL63 | S | 241 | GSTLVDGVSRLYQPL | 121 |
| NL63 | S | 386 | INGFKYFDLGFIEAV | 122 |
| NL63 | S | 396 | FIEAVNFNVTTASAT | 123 |
| NL63 | S | 416 | AFATFVDVLVNVSAT | 124 |
| NL63 | S | 466 | DNVLPETYVALPIYY | 125 |
| NL63 | S | 481 | QHTDINFTATASFGG | 126 |
| NL63 | S | 496 | SCYVCKPHQVNISLN | 127 |
| NL63 | S | 766 | PSNWTTSVQVEYLQI | 128 |
| NL63 | S | 771 | TSVQVEYLQITSTPI | 129 |
| NL63 | S | 781 | TSTPIVVDCATYVCN | 130 |
| NL63 | S | 786 | VVDCATYVCNGNPRC | 131 |
| NL63 | S | 796 | GNPRCKNLLKQYTSA | 132 |
| NL63 | S | 866 | RIAGRSALEDLLFSK | 133 |
| NL63 | S | 871 | SALEDLLFSKVVTSG | 134 |
| NL63 | S | 951 | SLALQARLNYVALQT | 135 |
| NL63 | S | 956 | ARLNYVALQTDVLQE | 136 |
| NL63 | S | 1051 | DSI QADQQVDRLITG | 137 |
| NL63 | S | 1056 | DQQVDRLITGRLAAL | 138 |
| NL63 | S | 1066 | RLAALNAFVSQVLNK | 139 |
| NL63 | S | 1071 | NAFVSQVLNKYTEVR | 140 |
| NL63 | S | 1081 | YTEVRGSRRLAQQKI | 141 |
| NL63 | S | 1086 | GSRRLAQQKINECVK | 142 |

TABLE 2
List of Additional CD4+ T cell epitopes.

| CCC | | | | SEQ ID |
|---|---|---|---|---|
| virus | Protein | Start | Sequence | NO: |
| NL63 | nsp2 | 126 | PVLPKNMWEFRDYFN | 143 |
| NL63 | nsp3 | 1756 | TCDICKSTVVEVKSA | 144 |
| NL63 | nsp3 | 1771 | IVCASVLKDGCDVGF | 145 |
| NL63 | nsp12 | 4491 | FDFYRYNKPTILDIC | 146 |
| NL63 | M | 46 | GLKMSVLWCLWPLVL | 147 |
| NL63 | M | 51 | VLWCLWPLVLALSIF | 148 |
| NL63 | M | 71 | FNVDWVFFGFSILMS | 149 |
| OC43 | nsp3 | 2566 | ANTGTSVTETMFDVY | 150 |
| OC43 | nsp12 | 4401 | LRAFDIYNASVAGIG | 151 |
| OC43 | nsp12 | 4426 | QRVDENGDKLDQFFV | 152 |
| OC43 | nsp12 | 4436 | DQFFVVKRTDLTIYN | 153 |
| OC43 | nsp12 | 4441 | VKRTDLTIYNREMKC | 154 |
| OC43 | nsp12 | 4451 | REMKCYERVKDCKFV | 155 |
| OC43 | nsp12 | 4601 | AIADSYYSYIMPMLT | 156 |
| OC43 | nsp12 | 4636 | VQYDFTDYKLELFNK | 157 |
| OC43 | nsp12 | 4706 | VVSIGYHYKELGIVM | 158 |
| OC43 | nsp12 | 4721 | NMDVDTHRYRLSLKD | 159 |
| OC43 | nsp12 | 4726 | THRYRLSLKDLLLYA | 160 |
| OC43 | nsp12 | 4731 | LSLKDLLLYAADPAL | 161 |
| OC43 | nsp12 | 5256 | LYNDLGNQILDSYSV | 162 |
| OC43 | nsp12 | 5296 | MQSVGACVVCSSQTS | 163 |
| OC43 | M | 196 | TSGFAVYVKSKVGNY | 164 |
| OC43 | N | 116 | QRQLLPRWYFYYLGT | 165 |
| OC43 | N | 151 | NQADVNTPADIVDRD | 166 |

It is an object of claims of the present invention to provide improved epitope or peptide combinations for modulating an immune response, for treating a subject for an infection or aberrant immune response, and for use in diagnostic methods and kits comprising such peptide combinations. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross reactivity to other viral strains, including co-circulating strains (for example, mutants) of coronaviruses, including SARS-CoV-2, common cold coronaviruses, as well as SARS-COV, MERS, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a SARS-COV-2 protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a coronavirus, including methods and compositions of detecting an immune response or immune cells relevant to a coronavirus infection. These methods and compositions include vaccines, diagnostics, therapies, reagents and kits, for modulating, eliciting, or detecting T cells responsive to one or more coronavirus peptides or proteins. The proteins and peptides described herein comprise, consist of, or consist essentially of: one or more amino acid sequences selected from those sequences set forth in Tables 1 or 2 (SEQ ID NOS: 1 to 166), such as 1, 2, 3, 4, 5, 6, 7, 8, 9, 10, 11, 12, 13, 14, 15, 16, 17, 18, 19, 20, 25, 30, 35, 40, 45, 50, 55, 60, 70, 75, 80, 90, 100, 125, 150, 160, or 166 peptides, or a subsequence, portion, homologue, variant or derivative thereof; a fusion protein comprising one or more amino acid sequences selected from those sequences set forth in Tables 1 or 2 (SEQ ID NOS: 1 to 166); a pool of 2 or more peptides selected from the amino acid sequences set forth in Tables 1 or 2 (SEQ ID NOS: 1 to 166), such as 2, 3, 4, 5, 6, 7, 8, 9, 10, 11, 12, 13, 14, 15, 16, 17, 18, 19, 20, 25, 30, 35, 40, 45, 50, 55, 60, 70, 75, 80, 90, 100, 125, 150, 160, or 166 peptides, or a polynucleotide that encodes one or more peptides or proteins, comprising, consisting of, or consisting essentially of an amino acid sequence selected from those sequences set forth in Tables 1 or 2 (SEQ ID NOS: 1 to 166), such as 1, 2, 3, 4, 5, 6, 7, 8, 9, 10, 11, 12, 13, 14, 15, 16, 17, 18, 19, 20, 25, 30, 35, 40, 45, 50, 55, 60, 70, 75, 80, 90, 100, 125, 150, 160, or 166 peptides, or a subsequence, portion, homologue, variant or derivative thereof. In certain preferred embodiments, the coronavirus is one or more of SARS-COV-2 or a variant thereof, or SARS, MERS, or a common cold coronavirus strain (e.g., 229E, NL63, HKU1, OC43). Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

The present inventors recognized that defining a comprehensive set of epitope specificities is important for several reasons. First, it allows the determination of whether within different SARS-COV-2 antigens certain regions are immunodominant. This will be important for vaccine design, so as to ensure that vaccine constructs include not only regions targeted by neutralizing antibodies, such as the receptor binding domain (RBD) in the spike (S) region, but also include regions capable of delivering sufficient T cell help and are suitable targets of CD4+ T cell activity. Second, a comprehensive set of epitopes helps define the breadth of responses, in terms of the average number of different CD4+ and CD8+ T cell SARS-COV-2 epitopes generally recognized by each individual. This is key because some reports have described a T cell repertoire focused on few viral epitopes (Ferretti et al., 2020), which would be concerning for potential viral escape from immune recognition via accumulated mutations that can occur during replication or through viral reassortment. Third, a comprehensive survey of epitopes restricted by a set of different HLAs representative of the diversity present in the general population is important to ensure that results obtained are generally applicable across different ethnicities and racial groups, and also to lay the foundations to examine the potential associations of certain HLAs with COVID-19 severity. Finally, the definition of the epitopes recognized in SARS-COV-2 infection is relevant in the context of the debate on the potential influence of SARS-COV-2 cross-reactivity with endemic “Common Cold” Coronaviruses (CCC) (Braun et al., 2020; Le Bert et al., 2020). Several studies have defined the repertoire of SARS-COV-2 epitopes recognized in unexposed individuals (Braun et al., 2020; Mateus et al., 2020; Nelde et al., 2020), but the correspondence between that repertoire and the epitope repertoire elicited by SARS-COV-2 infection has not been previously evaluated.

The emergence of several new SARS-COV-2 variants remains of immediate concern to the medical and scientific community. Several other Corona Viruses (CoV) are known to infect bats and other animal species and are of concern as they could jump to human hosts and foster a new pandemic. For all the above reasons it is important to explore vaccine concepts that might be able to provide large spectrum immunity against several CoV viral species. The inventors describe herein the discovery of T cell epitopes that provide broad protective immunity to multiple CoV species, and the use of those epitopes in various compositions, methods, kits, etc., as provided herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and

combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A composition comprising:
one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof;
a fusion protein comprising one or more amino acid sequences selected from those sequences set forth in Tables 1 or 2 (SEQ ID NOS: 1 to 166); or
a pool of 2 or more or more peptides comprising, consisting of, or consisting essentially of amino acid sequences selected from those sequences set forth in Tables 1 or 2 (SEQ ID NOS: 1 to 166); or
a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof.

2. The composition of claim 1, wherein at least one of:
the one or more peptides or proteins comprises, or wherein the fusion protein comprises 2 or more or more amino acid sequences selected from those sequences set forth in Tables 1 or 2 (SEQ ID NOS: 1 to 166), or a subsequence, portion, homologue, variant or derivative thereof;
the one or more peptides or proteins comprises a coronavirus T cell epitope;
the one or more peptides or proteins comprises a coronavirus CD8+ or CD4+ T cell epitope;
the one or more peptides or proteins comprises has a length from about 9-15, 15-20, 20-25, 25-30, 30-40, 40-50, 50-75 or 75-100 amino acids;
the one or more peptides or proteins comprises elicits, stimulates, induces, promotes, increases or enhances a T cell response to a coronavirus;
the one or more peptides or proteins comprises elicits, stimulates, induces, promotes, increases or enhances the T cell response to the coronavirus is a coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof;
the one or more peptides or proteins comprises a coronavirus T cell epitope selected from those sequences set forth in Tables 1 or 2 (SEQ ID NOS: 1 to 166);
the one or more peptides or proteins selected from those NL63 or OC43 coronavirus peptide sequences set forth in Tables 1 or 2 (SEQ ID NOS: 1 to 166), or a subsequence, portion, homologue, variant or derivative thereof.

3.-6. (canceled)

7. The composition of claim 1, wherein the coronavirus is NL63 or OC43 and the T cell epitope is conserved or not conserved in another coronavirus.

8.-11. (canceled)

12. The composition of claim 1, further comprising formulating the one or more peptides or proteins into an immunogenic formulation with an adjuvant, a modulator of immune response, a modulator of the innate immune response,
wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands; or
wherein the modulator is Interleukin-6 (IL-6), Interferon-gamma (IFN-γ), Transforming growth factor beta (TGF-β), or Interleukin-10 (IL-10), or an agonist or antagonist thereof.

13.-16. (canceled)

17. The composition of claim 1, further defined as comprising monomers or multimers of:
peptides or proteins comprising, consisting of, or consisting essentially of:
one or more amino acid sequences selected from those sequences set forth in Tables 1 or 2 (SEQ ID NOS: 1 to 166),
concatemers, subsequences, portions, homologues, variants or derivatives thereof;
a fusion protein comprising one or more amino acid sequences selected from those sequences set forth in Tables 1 or 2 (SEQ ID NOS: 1 to 166); or
a polynucleotide that encodes one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof.

18. The composition of claim 1, further defined as composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Tables 1 or 2 (SEQ ID NOS: 1 to 166), in a groove of the MHC monomer or multimer.

19.-35. (canceled)

36. The composition of claim 1, wherein the compositions include amino acid sequences from two or more coronavirus species.

37. A method for detecting the presence of: (i) a coronavirus or (ii) an immune response relevant to coronavirus infections, vaccines or therapies, including T cells responsive to one or more coronavirus peptides, comprising:
providing one or more proteins or peptides for detection of an amount or a relative amount of, and/or the activity of, and/or the state of antigen-specific T-cells;
contacting a biological sample suspected of having coronavirus-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in Tables 1 or 2 (SEQ ID NOS: 1 to 166), or comprise a pool of 2 or more or more amino acid sequences set forth in Tables 1 or 2 (SEQ ID NOS: 1 to 166).

38. The method of claim 37, wherein detecting the amount or a relative amount of, and/or activity of antigen-specific T-cells comprises one or more steps of identification or detection of the antigen-specific T-cells and measuring the amount of the antigen-specific T-cells.

39. The method of claim 37, wherein at least one of:
the one or more peptides or proteins comprises 2 or more amino acid sequences selected from those set forth in Tables 1 or 2 (SEQ ID NOS: 1 to 166);
the one or more peptides or proteins comprises a coronavirus T cell epitope;
the one or more peptides or proteins comprises a coronavirus CD8+ or CD4+ T cell epitope;
the one or more peptides or proteins comprises has a length from about 9-15, 15-20, 20-25, 25-30, 30-40, 40-50, 50-75 or 75-100 amino acids;
the one or more peptides or proteins comprises elicits, stimulates, induces, promotes, increases or enhances a T cell response to a coronavirus;
the one or more peptides or proteins comprises elicits, stimulates, induces, promotes, increases or enhances the T cell response to the coronavirus is a coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof;
the one or more peptides or proteins comprises a coronavirus T cell epitope selected from those sequences set forth in Tables 1 or 2 (SEQ ID NOS: 1 to 166);
the one or more peptides or proteins selected from those NL63 or OC43 coronavirus peptide sequences set forth in Tables 1 or 2 (SEQ ID NOS: 1 to 166), or a subsequence, portion, homologue, variant or derivative thereof.

40. The method of claim 37, wherein the detecting the amount or a relative amount of, and/or activity of antigen-specific T-cells comprises indirect detection and/or direct detection.

41. The method of claim 37, wherein the method of detecting an immune response relevant to the coronavirus comprises the following steps:
providing an MHC monomer or an MHC multimer;
contacting a population T-cells to the MHC monomer or MHC multimer; and
measuring the number, activity or state of T-cells specific for the MHC monomer or MHC multimer, the MHC monomer or MHC multimer comprises a protein or peptide of the coronavirus, or the protein or peptide comprises a CD8+ or CD4+ T cell epitope.

42.-43. (canceled)

44. The method of claim 43, wherein the T cell epitope is conserved or not conserved in another coronavirus.

45.-47. (canceled)

48. The method of claim 37, further comprising detecting the presence or amount of the one or more peptides in a biological sample, or a response thereto, which is diagnostic of a coronavirus infection, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

49. (canceled)

50. The method of claim 37, further comprising administering a treatment comprising the composition of claim 1 to the subject from which the biological sample was drawn that increases the amount or relative amount of, and/or activity of the antigen-specific T-cells.

51. The method of claim 37, further comprising for detecting the presence of: (i) SARS-COV-2 or (ii) an immune response relevant to SARS-COV-2 infections, vaccines or therapies, including T cells responsive to one or more SARS-COV-2 peptides, comprising:
providing one or more proteins or peptides for detection of an amount or a relative amount of, and/or the activity of, and/or the state of antigen-specific T-cells;
contacting a biological sample suspected of having SARS-COV-2-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in those sequences set forth in Tables 1 or 2 (SEQ ID NOS: 1 to 166), or comprise a pool of 2 or more amino acid sequences set forth in those sequences set forth in Tables 1 or 2 (SEQ ID NOS: 1 to 166).

52.-64. (canceled)

65. The method of claim 37, further comprising detecting a coronavirus infection or exposure in a subject, the method comprising, consisting of, or consisting essentially of:
contacting a biological sample from a subject with a composition of claim 1; and
determining if the composition elicits an immune response from the contacted cells, wherein the presence of an immune response indicates that the subject has been exposed to or infected with coronavirus.

66. The method of claim 65, wherein the sample comprises T cells, wherein the T cells are CD8+ or CD4+ T cells, and the response comprises inducing, increasing, promoting or stimulating anti-coronavirus activity of T cells.

67.-68. (canceled)

69. The method of claim 65, wherein the method further comprises at least one of:
determining whether the subject has been infected by or exposed to the coronavirus more than once by determining if the subject elicits a secondary T cell immune response profile that is different from a primary T cell immune response profile;
diagnosing a coronavirus infection or exposure in a subject, the method comprising contacting a biological sample from a subject with a composition of claim 1, and determining if the composition elicits a T cell immune response, wherein the T cell immune response identifies that the subject has been infected with or exposed to a coronavirus; or
conducting the method three or more days following the date of suspected infection by or exposure to a coronavirus.

70.-78. (canceled)

79. A kit for the detection of coronavirus, an immune response to coronavirus, or detection of SARS-COV-2 or an immune response to SARS-COV-2 in a subject comprising, consisting of or consisting essentially of:
one or more T cells that specifically detect the presence of:
one or more amino acid sequences selected from those sequences set forth in Tables 1 or 2 (SEQ ID NOS: 1 to 166), or a subsequence, portion, homologue, variant or derivative thereof; or
a fusion protein comprising one or more amino acid sequences selected from those sequences set forth in Tables 1 or 2 (SEQ ID NOS: 1 to 166); or
a pool of 2 or more or more peptides selected from the amino acid sequences set forth in Tables 1 or 2 (SEQ ID NOS: 1 to 166).

80. The kit of claim 79, wherein at least one of:
the one or more amino acid sequences are selected from a coronavirus T cell epitope set forth in Tables 1 or 2 (SEQ ID NOS: 1 to 166);
the one or more peptides or proteins comprises a coronavirus T cell epitope;
the one or more peptides or proteins comprises a coronavirus CD8+ or CD4+ T cell epitope;
the one or more peptides or proteins comprises has a length from about 9-15, 15-20, 20-25, 25-30, 30-40, 40-50, 50-75 or 75-100 amino acids;
the one or more peptides or proteins comprises elicits, stimulates, induces, promotes, increases or enhances a T cell response to a coronavirus;
the one or more peptides or proteins comprises elicits, stimulates, induces, promotes, increases or enhances the T cell response to the coronavirus is a coronavirus spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof;
the one or more peptides or proteins comprises a coronavirus T cell epitope selected from those sequences set forth in Tables 1 or 2 (SEQ ID NOS: 1 to 166);
the one or more peptides or proteins selected from those NL63 or OC43 coronavirus peptide sequences set forth in Tables 1 or 2 (SEQ ID NOS: 1 to 166), or a subsequence, portion, homologue, variant or derivative thereof.

81.-85. (canceled)

86. The kit of claim 79, wherein the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus.

87. The kit of claim 79, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

88. The kit of claim 79, wherein the kit includes reagents for determining a Human Leukocyte Antigen (HLA) profile of a subject, and selecting peptides that are presented by the HLA profile of the subject for detecting an immune response to coronavirus.

89.-95. (canceled)

96. The kit of claim 79, wherein the kit includes instruction for a diagnostic method, a process, a composition, a product, a service or component part thereof for the detection of: (i) SARS-COV-2 or (ii) an immune response relevant to SARS-COV-2 infections, vaccines or therapies, including T cells responsive to SARS-COV-2.

97.-98. (canceled)

99. A method of treating, preventing, immunizing, stimulating, inducing, promoting, increasing, or enhancing an immune response against a coronavirus or against SARS-COV-2, in a subject, comprising:
administering a composition of claim 1, in an amount sufficient to stimulate, induce, promote, increase, or enhance an immune response against the coronavirus in the subject.

100.-105. (canceled)

106. The method of claim 99, further comprising
administering to a subject an amount of a protein or peptide or a polynucleotide that expresses the protein or peptide comprising, consisting of or consisting essentially of an amino acid sequence of a NL63 or OC43 spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Tables 1 or 2 (SEQ ID NOS: 1 to 166), or a subsequence, portion, homologue, variant or derivative thereof, in an amount sufficient to prevent, stimulate, induce, promote, increase, immunize against, or enhance an immune response against SARS-COV-2 in the subject.

107.-120. (canceled)

121. The method of claim 99, wherein the protein or peptide, or a subsequence, portion, homologue, variant or derivative thereof is administered within 2-72 hours, 2-48 hours, 4-24 hours, 4-18 hours, or 6-12 hours after a symptom of SARS-COV-2 infection or exposure develops.

122.-137. (canceled)

138. A peptide or peptides that are immunoprevalent or immunodominant in a virus obtained by a method consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool.

139.-143. (canceled)

144. A method of selecting an immunoprevalent or immunodominant peptide or protein of a virus comprising, consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool.

145.-149. (canceled)

150. A polynucleotide that expresses one or more peptides or proteins, comprising, consisting of, or consisting essentially of an amino acid sequence selected from those sequences set forth in Tables 1 or 2 (SEQ ID NOS: 1 to 166), or a subsequence, portion, homologue, variant or derivative thereof;
a fusion protein comprising one or more amino acid sequences selected from those sequences set forth in Tables 1 or 2 (SEQ ID NOS: 1 to 166);
a pool of 2 or more or more peptides comprising, consisting of, or consisting essentially of amino acid sequences selected from those sequences set forth in Tables 1 or 2 (SEQ ID NOS: 1 to 166);
one or more peptides or proteins comprising, consisting of, or consisting essentially of an amino acid sequence selected from those sequences set forth in Tables 1 or 2 (SEQ ID NOS: 1 to 166), or a subsequence, portion, homologue, variant or derivative thereof;
a fusion protein comprising one or more amino acid sequences selected from those sequences set forth in Tables 1 or 2 (SEQ ID NOS: 1 to 166); or
a pool of 2 or more peptides selected from those sequences set forth in Tables 1 or 2 (SEQ ID NOS: 1 to 166)

151. The polynucleotide of claim 150, wherein the polynucleotide is in a vector or a viral vector.

152. (canceled)

153. The polynucleotide of claim 150, wherein the polynucleotide is in a vector that is in a host cell.

154.-157. (canceled)


SEQUENCE LISTING (166 sequences)

SEQ ID NO: 1 - protein, 15 aa, synthetic construct
  REGION 1..15: note = Synthetic
DGLEAYADKTLQEMK

SEQ ID NO: 2 - protein, 15 aa, synthetic construct
  REGION 1..15: note = Synthetic
LQEMKALFPTWSQEL

SEQ ID NO: 3 - protein, 15 aa, synthetic construct
  REGION 1..15: note = Synthetic
LFDVIVAWHVVRDPR

SEQ ID NO: 4 - protein, 15 aa, synthetic construct
  REGION 1..15: note = Synthetic
KFSLETFTVCADGFM

SEQ ID NO: 5 - protein, 15 aa, synthetic construct
  REGION 1..15: note = Synthetic
LSCFKIGRRRICLSG

SEQ ID NO: 6 - protein, 15 aa, synthetic construct
  REGION 1..15: note = Synthetic
ICLSGRKIYEVERGL

SEQ ID NO: 7 - protein, 15 aa, synthetic construct
  REGION 1..15: note = Synthetic
VERGLLHSSQLPLDV

SEQ ID NO: 8 - protein, 15 aa, synthetic construct
  REGION 1..15: note = Synthetic
EVLEDTLDDGPSVET

SEQ ID NO: 9 - protein, 15 aa, synthetic construct
  REGION 1..15: note = Synthetic
NVCFVKGDIIKVSKL

SEQ ID NO: 10 - protein, 15 aa, synthetic construct
  REGION 1..15: note = Synthetic
RTVKYFECTGGIDIC

SEQ ID NO: 11 - protein, 15 aa, synthetic construct
  REGION 1..15: note = Synthetic
FIGDKVGHYVHVKCE

SEQ ID NO: 12 - protein, 15 aa, synthetic construct
  REGION 1..15: note = Synthetic
HVKCEQSYQLYDASN

SEQ ID NO: 13 - protein, 15 aa, synthetic construct
  REGION 1..15: note = Synthetic
YDASNVKKVTDVTGK

SEQ ID NO: 14 - protein, 15 aa, synthetic construct
  REGION 1..15: note = Synthetic
LLFGWIKISADNKVI

SEQ ID NO: 15 - protein, 15 aa, synthetic construct
  REGION 1..15: note = Synthetic
YTTEIASKLTCKLVA

SEQ ID NO: 16 - protein, 15 aa, synthetic construct
  REGION 1..15: note = Synthetic
NVIFSDFYLPKIGFL

SEQ ID NO: 17 - protein, 15 aa, synthetic construct
  REGION 1..15: note = Synthetic
DFYLPKIGFLPTFVG

SEQ ID NO: 18 - protein, 15 aa, synthetic construct
  REGION 1..15: note = Synthetic
PTFVGKIAQWIKNTF

SEQ ID NO: 19 - protein, 15 aa, synthetic construct
  REGION 1..15: note = Synthetic
NLITTANTGTSVTET

SEQ ID NO: 20 - protein, 15 aa, synthetic construct
  REGION 1..15: note = Synthetic
ANTGTSVTETMFDVY

SEQ ID NO: 21 - protein, 15 aa, synthetic construct
  REGION 1..15: note = Synthetic
APQANSGDYYYKVEA

SEQ ID NO: 22 - protein, 15 aa, synthetic construct
  REGION 1..15: note = Synthetic
FLKEWNFSLGIILLF

SEQ ID NO: 23 - protein, 15 aa, synthetic construct
  REGION 1..15: note = Synthetic
ITIILQFGYTSRSMF

SEQ ID NO: 24 - protein, 15 aa, synthetic construct
  REGION 1..15: note = Synthetic
SRSMFVYVIKMIILW

SEQ ID NO: 25 - protein, 15 aa, synthetic construct
  REGION 1..15: note = Synthetic
MIILWLMWPLTIILT

SEQ ID NO: 26 - protein, 15 aa, synthetic construct
  REGION 1..15: note = Synthetic
IVAIIMWIVYFVNSI

SEQ ID NO: 27 - protein, 15 aa, synthetic construct
  REGION 1..15: note = Synthetic
FVNSIRLFIRTGSFW

SEQ ID NO: 28 - protein, 15 aa, synthetic construct
  REGION 1..15: note = Synthetic
TGSFWSFNPETNNLM

SEQ ID NO: 29 - protein, 15 aa, synthetic construct
  REGION 1..15: note = Synthetic
RPIIEDYHTLTVTII

SEQ ID NO: 30 - protein, 15 aa, synthetic construct
  REGION 1..15: note = Synthetic
TVTIIRGHLYIQGIK

SEQ ID NO: 31 - protein, 15 aa, synthetic construct
  REGION 1..15: note = Synthetic
IQGIKLGTGYSLADL

SEQ ID NO: 32 - protein, 15 aa, synthetic construct
  REGION 1..15: note = Synthetic
SLADLPAYMTVAKVT

SEQ ID NO: 33 - protein, 15 aa, synthetic construct
  REGION 1..15: note = Synthetic
QKGSGMDTALLRNNI

SEQ ID NO: 34 - protein, 15 aa, synthetic construct
  REGION 1..15: note = Synthetic
DQFRNVQTRGRRAQP

SEQ ID NO: 35 - protein, 15 aa, synthetic construct
  REGION 1..15: note = Synthetic
FSGITQFQKGKEFEF

SEQ ID NO: 36 - protein, 15 aa, synthetic construct
  REGION 1..15: note = Synthetic
PRWYFYYLGTGPHAK

SEQ ID NO: 37 - protein, 15 aa, synthetic construct
  REGION 1..15: note = Synthetic
YYLGTGPHAKDQYGT

SEQ ID NO: 38 - protein, 15 aa, synthetic construct
  REGION 1..15: note = Synthetic
DIDGVYWVASNQADV

SEQ ID NO: 39 - protein, 15 aa, synthetic construct
  REGION 1..15: note = Synthetic
NLSGNPDEPQKDVYE

SEQ ID NO: 40 - protein, 15 aa, synthetic construct
  REGION 1..15: note = Synthetic
KDVYELRYNGAIRFD

SEQ ID NO: 41 - protein, 15 aa, synthetic construct
  REGION 1..15: note = Synthetic
LRYNGAIRFDSTLSG

SEQ ID NO: 42 - protein, 15 aa, synthetic construct
  REGION 1..15: note = Synthetic
AIRFDSTLSGFETIM

SEQ ID NO: 43 - protein, 15 aa, synthetic construct
  REGION 1..15: note = Synthetic
STLSGFETIMKVLNE

SEQ ID NO: 44 - protein, 15 aa, synthetic construct
  REGION 1..15: note = Synthetic
FETIMKVLNENLNAY

SEQ ID NO: 45 - protein, 15 aa, synthetic construct
  REGION 1..15: note = Synthetic
NLNAYQQQDGMMNMS

SEQ ID NO: 46 - protein, 15 aa, synthetic construct
  REGION 1..15: note = Synthetic
LKKMDEPYTEDTSEI

SEQ ID NO: 47 - protein, 15 aa, synthetic construct
  REGION 1..15: note = Synthetic
NTKVIKDRVMYSEFP

SEQ ID NO: 48 - protein, 15 aa, synthetic construct
  REGION 1..15: note = Synthetic
TIDKFAIPNGRKVDL

SEQ ID NO: 49 - protein, 15 aa, synthetic construct
  REGION 1..15: note = Synthetic
NTDIILGVCVNYDLY

SEQ ID NO: 50 - protein, 15 aa, synthetic construct
  REGION 1..15: note = Synthetic
NLLYDSNGNLYGFRD

SEQ ID NO: 51 - protein, 15 aa, synthetic construct
  REGION 1..15: note = Synthetic
TRQLQPINYFDSYLG

SEQ ID NO: 52 - protein, 15 aa, synthetic construct
  REGION 1..15: note = Synthetic
PINYFDSYLGCVVNA

SEQ ID NO: 53 - protein, 15 aa, synthetic construct
  REGION 1..15: note = Synthetic
NFNVDDINFSPVLGC

SEQ ID NO: 54 - protein, 15 aa, synthetic construct
  REGION 1..15: note = Synthetic
RSAIEDLLFDKVKLS

SEQ ID NO: 55 - protein, 15 aa, synthetic construct
  REGION 1..15: note = Synthetic
DRLINGRLTALNAYV

SEQ ID NO: 56 - protein, 15 aa, synthetic construct
  REGION 1..15: note = Synthetic
GRLTALNAYVSQQLS

SEQ ID NO: 57 - protein, 15 aa, synthetic construct
  REGION 1..15: note = Synthetic
LNAYVSQQLSDSTLV

SEQ ID NO: 58 - protein, 15 aa, synthetic construct
  REGION 1..15: note = Synthetic
SQQLSDSTLVKFSAA

SEQ ID NO: 59 - protein, 15 aa, synthetic construct
  REGION 1..15: note = Synthetic
DSTLVKFSAAQAMEK

SEQ ID NO: 60 - protein, 15 aa, synthetic construct
  REGION 1..15: note = Synthetic
QNAPYGLYFIHFSYV

SEQ ID NO: 61 - protein, 15 aa, synthetic construct
  REGION 1..15: note = Synthetic
LNTSIPNLPDFKEEL

SEQ ID NO: 62 - protein, 15 aa, synthetic construct
  REGION 1..15: note = Synthetic
DQWFKNQTSVAPDLS

SEQ ID NO: 63 - protein, 15 aa, synthetic construct
  REGION 1..15: note = Synthetic
SAMNVIGQHIKLPQF

SEQ ID NO: 64 - protein, 15 aa, synthetic construct
  REGION 1..15: note = Synthetic
VSKPVMISQWPISND

SEQ ID NO: 65 - protein, 15 aa, synthetic construct
  REGION 1..15: note = Synthetic
MISQWPISNDSNGCV

SEQ ID NO: 66 - protein, 15 aa, synthetic construct
  REGION 1..15: note = Synthetic
DSVREEVDIIEQPFE

SEQ ID NO: 67 - protein, 15 aa, synthetic construct
  REGION 1..15: note = Synthetic
GVRVLDQSDNNCWIS

SEQ ID NO: 68 - protein, 15 aa, synthetic construct
  REGION 1..15: note = Synthetic
NCWISTTLVQLQLTK

SEQ ID NO: 69 - protein, 15 aa, synthetic construct
  REGION 1..15: note = Synthetic
TTLVQLQLTKLLDDS

SEQ ID NO: 70 - protein, 15 aa, synthetic construct
  REGION 1..15: note = Synthetic
LLDDSIEMQLFKVGK

SEQ ID NO: 71 - protein, 15 aa, synthetic construct
  REGION 1..15: note = Synthetic
VDSIVQKCYELSHLI

SEQ ID NO: 72 - protein, 15 aa, synthetic construct
  REGION 1..15: note = Synthetic
EIEAGEVKPFAVYKN

SEQ ID NO: 73 - protein, 15 aa, synthetic construct
  REGION 1..15: note = Synthetic
VNAANENLLHGGGVA

SEQ ID NO: 74 - protein, 15 aa, synthetic construct
  REGION 1..15: note = Synthetic
KVGAGVMLECEKFNV

SEQ ID NO: 75 - protein, 15 aa, synthetic construct
  REGION 1..15: note = Synthetic
VMLECEKFNVFNVVG

SEQ ID NO: 76 - protein, 15 aa, synthetic construct
  REGION 1..15: note = Synthetic
DLTPVIDDVDVVKPF

SEQ ID NO: 77 - protein, 15 aa, synthetic construct
  REGION 1..15: note = Synthetic
RVEGNFSFFDCGVNA

SEQ ID NO: 78 - protein, 15 aa, synthetic construct
  REGION 1..15: note = Synthetic
LDGDIYLLFTNSILM

SEQ ID NO: 79 - protein, 15 aa, synthetic construct
  REGION 1..15: note = Synthetic
PAIDVLKKLLSSLTL

SEQ ID NO: 80 - protein, 15 aa, synthetic construct
  REGION 1..15: note = Synthetic
LKKLLSSLTLTVKFV

SEQ ID NO: 81 - protein, 15 aa, synthetic construct
  REGION 1..15: note = Synthetic
TVKFVVESNVMDVND

SEQ ID NO: 82 - protein, 15 aa, synthetic construct
  REGION 1..15: note = Synthetic
FYVNANGGTCFCNKH

SEQ ID NO: 83 - protein, 15 aa, synthetic construct
  REGION 1..15: note = Synthetic
FCNKHNFFCVNCDSF

SEQ ID NO: 84 - protein, 15 aa, synthetic construct
  REGION 1..15: note = Synthetic
RFKNADLKDGYFVIK

SEQ ID NO: 85 - protein, 15 aa, synthetic construct
  REGION 1..15: note = Synthetic
HDFFTWKDGRVIYGN

SEQ ID NO: 86 - protein, 15 aa, synthetic construct
  REGION 1..15: note = Synthetic
SKGWYDPVENEDIHR

SEQ ID NO: 87 - protein, 15 aa, synthetic construct
  REGION 1..15: note = Synthetic
GKIVARAMLKCVALC

SEQ ID NO: 88 - protein, 15 aa, synthetic construct
  REGION 1..15: note = Synthetic
KGVVGVLTLDNQDLN

SEQ ID NO: 89 - protein, 15 aa, synthetic construct
  REGION 1..15: note = Synthetic
KHFFFAQNGDAAVKD

SEQ ID NO: 90 - protein, 15 aa, synthetic construct
  REGION 1..15: note = Synthetic
YLPYPDPSRILSAGV

SEQ ID NO: 91 - protein, 15 aa, synthetic construct
  REGION 1..15: note = Synthetic
VKTDAVVLLERYVSL

SEQ ID NO: 92 - protein, 15 aa, synthetic construct
  REGION 1..15: note = Synthetic
VVLLERYVSLAIDAY

SEQ ID NO: 93 - protein, 15 aa, synthetic construct
  REGION 1..15: note = Synthetic
RYVSLAIDAYPLSKH

SEQ ID NO: 94 - protein, 15 aa, synthetic construct
  REGION 1..15: note = Synthetic
VPLLEVYVHLRNWNF

SEQ ID NO: 95 - protein, 15 aa, synthetic construct
  REGION 1..15: note = Synthetic
VYVHLRNWNFSWNLI

SEQ ID NO: 96 - protein, 15 aa, synthetic construct
  REGION 1..15: note = Synthetic
RNWNFSWNLILTLFI

SEQ ID NO: 97 - protein, 15 aa, synthetic construct
  REGION 1..15: note = Synthetic
SWNLILTLFIVVLQY

SEQ ID NO: 98 - protein, 15 aa, synthetic construct
  REGION 1..15: note = Synthetic
LTLFIVVLQYGHYKY

SEQ ID NO: 99 - protein, 15 aa, synthetic construct
  REGION 1..15: note = Synthetic
VVLQYGHYKYSRLLY

SEQ ID NO: 100 - protein, 15 aa, synthetic construct
  REGION 1..15: note = Synthetic
GHYKYSRLLYGLKMS

SEQ ID NO: 101 - protein, 15 aa, synthetic construct
  REGION 1..15: note = Synthetic
WPLVLALSIFDCFVN

SEQ ID NO: 102 - protein, 15 aa, synthetic construct
  REGION 1..15: note = Synthetic
DCFVNFNVDWVFFGF

SEQ ID NO: 103 - protein, 15 aa, synthetic construct
  REGION 1..15: note = Synthetic
RLWRRVKTFWAFNPE

SEQ ID NO: 104 - protein, 15 aa, synthetic construct
  REGION 1..15: note = Synthetic
LPVMAAPTGVTLTLL

SEQ ID NO: 105 - protein, 15 aa, synthetic construct
  REGION 1..15: note = Synthetic
SGVLLVDGHKIATRV

SEQ ID NO: 106 - protein, 15 aa, synthetic construct
  REGION 1..15: note = Synthetic
VDGHKIATRVQVGQL

SEQ ID NO: 107 - protein, 15 aa, synthetic construct
  REGION 1..15: note = Synthetic
QVGQLPKYVIVATPS

SEQ ID NO: 108 - protein, 15 aa, synthetic construct
  REGION 1..15: note = Synthetic
PKYVIVATPSTTIVC

SEQ ID NO: 109 - protein, 15 aa, synthetic construct
  REGION 1..15: note = Synthetic
TTIVCDRVGRSVNET

SEQ ID NO: 110 - protein, 15 aa, synthetic construct
  REGION 1..15: note = Synthetic
DRVGRSVNETSQTGW

SEQ ID NO: 111 - protein, 15 aa, synthetic construct
  REGION 1..15: note = Synthetic
SVNETSQTGWAFYVR

SEQ ID NO: 112 - protein, 15 aa, synthetic construct
  REGION 1..15: note = Synthetic
AKHGDFSGVASQEGV

SEQ ID NO: 113 - protein, 15 aa, synthetic construct
  REGION 1..15: note = Synthetic
FPPPSFYMPLLVSSD

SEQ ID NO: 114 - protein, 15 aa, synthetic construct
  REGION 1..15: note = Synthetic
FYMPLLVSSDKAPYR

SEQ ID NO: 115 - protein, 15 aa, synthetic construct
  REGION 1..15: note = Synthetic
VAKEGAKTVNTSLGN

SEQ ID NO: 116 - protein, 15 aa, synthetic construct
  REGION 1..15: note = Synthetic
SSSDLVAAVTLALKN

SEQ ID NO: 117 - protein, 15 aa, synthetic construct
  REGION 1..15: note = Synthetic
VAAVTLALKNLGFDN

SEQ ID NO: 118 - protein, 15 aa, synthetic construct
  REGION 1..15: note = Synthetic
YTYKMLVAKDNKNLP

SEQ ID NO: 119 - protein, 15 aa, synthetic construct
  REGION 1..15: note = Synthetic
NKNLPKFIEQISAFT

SEQ ID NO: 120 - protein, 15 aa, synthetic construct
  REGION 1..15: note = Synthetic
IPNGFPFNNWFLLTN

SEQ ID NO: 121 - protein, 15 aa, synthetic construct
  REGION 1..15: note = Synthetic
GSTLVDGVSRLYQPL

SEQ ID NO: 122 - protein, 15 aa, synthetic construct
  REGION 1..15: note = Synthetic
INGFKYFDLGFIEAV

SEQ ID NO: 123 - protein, 15 aa, synthetic construct
  REGION 1..15: note = Synthetic
FIEAVNFNVTTASAT

SEQ ID NO: 124 - protein, 15 aa, synthetic construct
  REGION 1..15: note = Synthetic
AFATFVDVLVNVSAT

SEQ ID NO: 125 - protein, 15 aa, synthetic construct
  REGION 1..15: note = Synthetic
DNVLPETYVALPIYY

SEQ ID NO: 126 - protein, 15 aa, synthetic construct
  REGION 1..15: note = Synthetic
QHTDINFTATASFGG

SEQ ID NO: 127 - protein, 15 aa, synthetic construct
  REGION 1..15: note = Synthetic
SCYVCKPHQVNISLN

SEQ ID NO: 128 - protein, 15 aa, synthetic construct
  REGION 1..15: note = Synthetic
PSNWTTSVQVEYLQI

SEQ ID NO: 129 - protein, 15 aa, synthetic construct
  REGION 1..15: note = Synthetic
TSVQVEYLQITSTPI

SEQ ID NO: 130 - protein, 15 aa, synthetic construct
  REGION 1..15: note = Synthetic
TSTPIVVDCATYVCN

SEQ ID NO: 131 - protein, 15 aa, synthetic construct
  REGION 1..15: note = Synthetic
VVDCATYVCNGNPRC

SEQ ID NO: 132 - protein, 15 aa, synthetic construct
  REGION 1..15: note = Synthetic
GNPRCKNLLKQYTSA

SEQ ID NO: 133 - protein, 15 aa, synthetic construct
  REGION 1..15: note = Synthetic
RIAGRSALEDLLFSK

SEQ ID NO: 134 - protein, 15 aa, synthetic construct
  REGION 1..15: note = Synthetic
SALEDLLFSKVVTSG

SEQ ID NO: 135 - protein, 15 aa, synthetic construct
  REGION 1..15: note = Synthetic
SLALQARLNYVALQT

SEQ ID NO: 136 - protein, 15 aa, synthetic construct
  REGION 1..15: note = Synthetic
ARLNYVALQTDVLQE

SEQ ID NO: 137 - protein, 15 aa, synthetic construct
  REGION 1..15: note = Synthetic
DSIQADQQVDRLITG

SEQ ID NO: 138 - protein, 15 aa, synthetic construct
  REGION 1..15: note = Synthetic
DQQVDRLITGRLAAL

SEQ ID NO: 139 - protein, 15 aa, synthetic construct
  REGION 1..15: note = Synthetic
RLAALNAFVSQVLNK

SEQ ID NO: 140 - protein, 15 aa, synthetic construct
  REGION 1..15: note = Synthetic
NAFVSQVLNKYTEVR

SEQ ID NO: 141 - protein, 15 aa, synthetic construct
  REGION 1..15: note = Synthetic
YTEVRGSRRLAQQKI

SEQ ID NO: 142 - protein, 15 aa, synthetic construct
  REGION 1..15: note = Synthetic
GSRRLAQQKINECVK

SEQ ID NO: 143 - protein, 15 aa, synthetic construct
  REGION 1..15: note = Synthetic
PVLPKNMWEFRDYFN

SEQ ID NO: 144 - protein, 15 aa, synthetic construct
  REGION 1..15: note = Synthetic
TCDICKSTVVEVKSA

SEQ ID NO: 145 - protein, 15 aa, synthetic construct
  REGION 1..15: note = Synthetic
IVCASVLKDGCDVGF

SEQ ID NO: 146 - protein, 15 aa, synthetic construct
  REGION 1..15: note = Synthetic
FDFYRYNKPTILDIC

SEQ ID NO: 147 - protein, 15 aa, synthetic construct
  REGION 1..15: note = Synthetic
GLKMSVLWCLWPLVL

SEQ ID NO: 148 - protein, 15 aa, synthetic construct
  REGION 1..15: note = Synthetic
VLWCLWPLVLALSIF

SEQ ID NO: 149 - protein, 15 aa, synthetic construct
  REGION 1..15: note = Synthetic
FNVDWVFFGFSILMS

SEQ ID NO: 150 - protein, 15 aa, synthetic construct
  REGION 1..15: note = Synthetic
ANTGTSVTETMFDVY

SEQ ID NO: 151 - protein, 15 aa, synthetic construct
  REGION 1..15: note = Synthetic
LRAFDIYNASVAGIG

SEQ ID NO: 152 - protein, 15 aa, synthetic construct
  REGION 1..15: note = Synthetic
QRVDENGDKLDQFFV

SEQ ID NO: 153 - protein, 15 aa, synthetic construct
  REGION 1..15: note = Synthetic
DQFFVVKRTDLTIYN

SEQ ID NO: 154 - protein, 15 aa, synthetic construct
  REGION 1..15: note = Synthetic
VKRTDLTIYNREMKC

SEQ ID NO: 155 - protein, 15 aa, synthetic construct
  REGION 1..15: note = Synthetic
REMKCYERVKDCKFV

SEQ ID NO: 156 - protein, 15 aa, synthetic construct
  REGION 1..15: note = Synthetic
AIADSYYSYIMPMLT

SEQ ID NO: 157 - protein, 15 aa, synthetic construct
  REGION 1..15: note = Synthetic
VQYDFTDYKLELFNK

SEQ ID NO: 158 - protein, 15 aa, synthetic construct
  REGION 1..15: note = Synthetic
VVSIGYHYKELGIVM

SEQ ID NO: 159 - protein, 15 aa, synthetic construct
  REGION 1..15: note = Synthetic
NMDVDTHRYRLSLKD

SEQ ID NO: 160 - protein, 15 aa, synthetic construct
  REGION 1..15: note = Synthetic
THRYRLSLKDLLLYA

SEQ ID NO: 161 - protein, 15 aa, synthetic construct
  REGION 1..15: note = Synthetic
LSLKDLLLYAADPAL

SEQ ID NO: 162 - protein, 15 aa, synthetic construct
  REGION 1..15: note = Synthetic
LYNDLGNQILDSYSV

SEQ ID NO: 163 - protein, 15 aa, synthetic construct
  REGION 1..15: note = Synthetic
MQSVGACVVCSSQTS

SEQ ID NO: 164 - protein, 15 aa, synthetic construct
  REGION 1..15: note = Synthetic
TSGFAVYVKSKVGNY

SEQ ID NO: 165 - protein, 15 aa, synthetic construct
  REGION 1..15: note = Synthetic
QRQLLPRWYFYYLGT

SEQ ID NO: 166 - protein, 15 aa, synthetic construct
  REGION 1..15: note = Synthetic
NQADVNTPADIVDRD